(19) United States
(12) Patent Application Publication
(10) Pub. No.: US 2011/0009629 A1
(43) Pub. Date: Jan. 13, 2011

(54) PREPARATION OF MORPHOLINE DERIVATIVES

(75) Inventors: Martin Albert, Kundl / Tirol (AT); Dominic DeSouza, Kundl / Tirol (AT); Kerstin Knepper, Kundl / Tirol (AT)

Correspondence Address: Manelii Denison & Selter PLLC, 2000 M Street, 7th Floor, Washington DC, DC, 20036, US

(73) Assignee: SANDOZ AG, Basel (CH)

(21) Appl. No.: 12/919,430

(22) PCT Filed: Feb. 20, 2009
(86) PCT No.: PCT/EP2009/052036

§ 371 Date: Aug. 30, 2010

(30) Foreign Application Priority Data
Feb. 26, 2008 (EP) 08151949.8

(51) Int. Cl. C07F 9/6558; C07D 413/06; C07D 265/32

(52) U.S. Cl. 544/132; 544/173; 544/174

(57) ABSTRACT

This invention relates to processes and intermediates for the stereoselective morpholine derivatives. The invention in particular allows the stereoselective preparation of the drugs aprepitant and fosaprepitant.

### FIELD OF INVENTION

This invention relates to processes and intermediates for the stereoselective synthesis of substituted morpholine derivatives, which can serve as NK₁ receptor antagonists. In particular, the invention allows the stereoselective preparation of new compounds, which allow an efficient access to aprepitant and fosaprepitant, two potent and orally active NK₁ antagonists.

### BACKGROUND OF THE INVENTION

Aprepitant (compound I; FIG. 1) has been first disclosed in EP 0734381 B1 and is currently being marketed as a treatment for chemotherapy-induced nausea and vomiting under the trade name Emend. In EP 0734381 B1 a synthetic route for this compound and a series of other morpholine derivatives is described. However, the disclosed processes for these compounds suffer from lengthy syntheses and low efficiency, which limits their use on industrial scale.

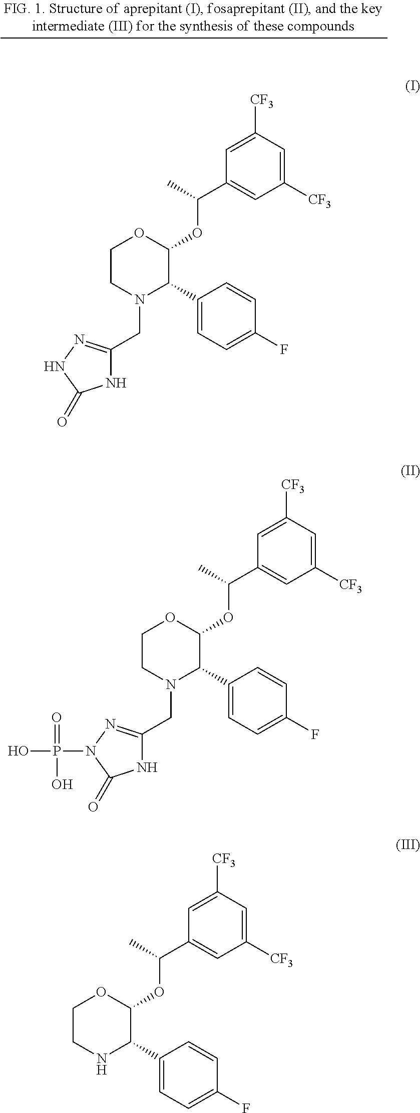

In EP 0748320 B1 the structure of fosaprepitant (compound II, FIG. 1) is disclosed. The preparation is based on an appropriate phosporyl transfer to aprepitant, optionally followed by a removal of phosphoryl protecting groups. In WO 99065900 A1 an improved process for the introduction of the 1,2,4-triazolin-5-on-yl-methyl side chain by using 3-chloromethyl-1,2,4-triazolin-5-one as alkylating agent is described. The novel process enables a one pot—one step process for the introduction of the side chain, thereby improving the prior art one pot—two step process. In WO 2003089429 A1 the prior art process for the introduction of the side chain is improved by conducting step 2 of the introduction of the side chain at a temperature ranging from 140° C. to 150° C.

An improved stereoselective synthesis for the key intermediate of formula III (FIG. 1) is disclosed in WO2001096319 A1. The process makes use of a chiral auxiliary attached to the nitrogen of the morpholine ring. A major drawback of this route is that the chiral auxiliary is destroyed during removal thus making the process expensive.

Another synthesis of key intermediate of formula III is described in WO 2001096320 A1. This route makes use of an intramolecular rearrangement to correctly set the hard to make acetal center. The stereochemistry of the exocyclic methyl group is established by a prior art process, giving an unfavorable mixture of isomers at this center.

U.S. Pat. No. 5,668,280 A and U.S. Pat. No. 6,130,331 A disclose approaches for the synthesis of key intermediate of formula III based on crystallization induced diastereomeric transformations. In both patents a selective reduction of a lacton using an expensive metal hydride is required.

In WO2007044829 A2 a mixture of 4 of 8 possible stereoisomers is prepared in an unselective manner. The desired isomer is isolated by several purification steps from this mixture of isomers, resulting in a low overall yield.

In WO 2001094322 A1, WO 2001094323 A1, and WO 2001094324 A1 an efficient process for the synthesis of the key intermediate of formula III by making use of a crystallization induced diastereomeric transformation is described. In this process the isolation of only three intermediates en route to aprepitant is required, making this process interesting from an economical point of view.

Although some efficient processes for the synthesis of aprepitant and fosaprepitant are available, further improvements in terms of number of isolated intermediates and overall yield would be highly desirable. In the following such an improved process is described. We have found, that a very efficient three component coupling, which allows the construction of the morpholine core in one step, can be combined with a selective crystallization with a chiral acid, which allows to establish the required stereochemistry. The chiral acid can easily be recovered and reused; the undesired isomers can be recycled by racemization.

En route to aprepitant only two intermediates need to be isolated, making the described process highly economical.

### THE INVENTION

The present invention relates to a process for the asymmetric synthesis of aprepitant or fosaprepitant, comprising the steps of

a) a three component coupling of an amino alcohol of formula VI, of 4-fluorophenylboronic acid or a C₁₋₆ alkyl or cyclic ester thereof (formula V), and of glyoxal (IV). Crystallization of the obtained morpholine derivative as addition salt with a chiral acid; isolation of a product of formula VII.chiral acid. Optionally, the undesired isomers in the mother liquor are racemized by treatment with acid or base, optionally at higher temperature, and the racemic morpholine derivative is resubjected to a crystallization with a chiral acid.

Optionally, the racemization of the undesired isomers and the crystallization of the desired isomer as a chiral acid addition salt is performed in a one pot fashion;

b) generation of hemi-acetal VII free base by portioning compound of formula VII.chiral acid between an alkaline aqueous layer and a water-immiscible organic phase; activation of the hemi-acetal functionality by transforming the OH-group into an activated derivative; reaction of the resulting activated acetal with alcohol of formula VIII. Removal of impurities by extraction to get a solution of compound IX;

c) removal of the N-protecting group to give the amine of formula X;

d) oxidation of the amine of formula X to the corresponding cyclic imine of formula XI; isolation of the cyclic imine of formula XI;

e) reduction of imine XI with a catalyst and H₂ or an H₂ equivalent; removal of the catalyst by filtration to get the key intermediate of formula III;

f) alkylation of compound of formula III to give aprepitant or fosaprepitant directly or via e.g. protected intermediates;

g) optionally, conversion of aprepitant to fosaprepitant by phosphorylation or a phosphorylation—deprotection sequence.

Scheme 1 exemplifies the process of the present invention by way of a sequence of isolated and non-isolated intermediates. Optionally, more intermediates can be isolated. One aspect of this invention is, that it is possible to remove undesired diasteoreomers by isolating the compounds of formula VII and XI. These compounds are particularly suited for this purpose (good depletion of undesired compounds during crystallization) resulting in a I or II with high purity.

The process of the invention has the advantage that it is fast, economic, simple, and produces aprepitant and fosaprepitant, respectively, in high yield and high optical purity.

The invention further relates to the new compounds of formula VII in crystalline form and to diastereomers of formula XI, I, and II.

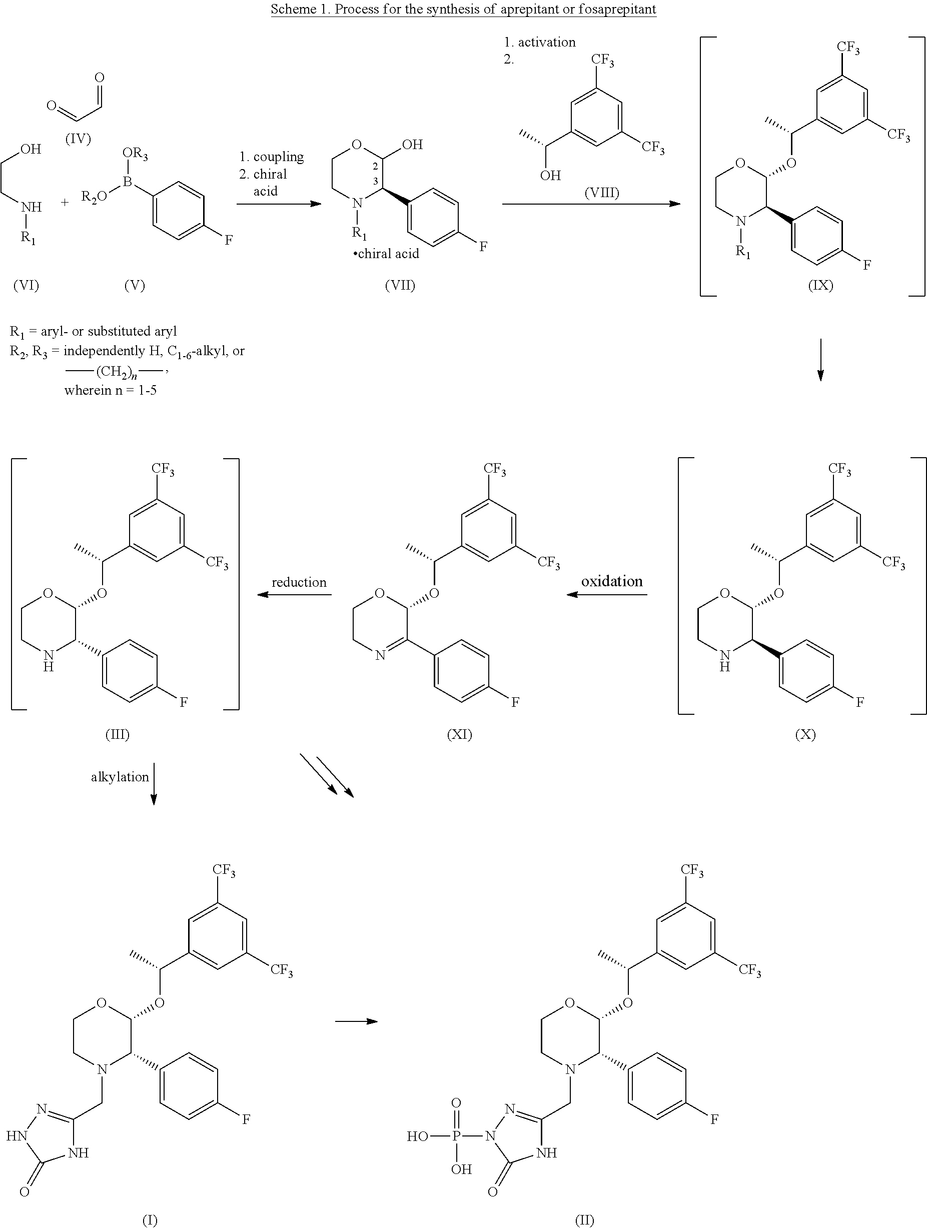

Alternatively, a compound of formula iso-VII may be isolated as an addition salt with a chiral acid and, following the same reaction sequence as for the preparation of aprepitant or fosaprepitant, the synthesis of isomers of aprepitant or fosaprepitant, usually present as impurities in the synthesis of aprepitant or fosaprepitant is also available.

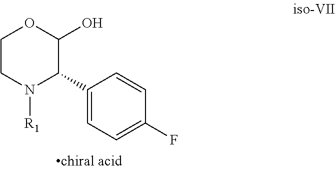

### DETAILED DESCRIPTION OF THE INVENTION

The present invention relates to a process for the asymmetric synthesis of morpholine derivatives, preferably to aprepitant and fosaprepitant comprising the steps of

a) a three component coupling of an amino alcohol of formula VI, of 4-fluorophenylboronic acid or a C₁₋₆ alkyl or cyclic ester thereof (formula V), and of glyoxal (IV). Crystallization of the obtained morpholine derivative as addition salt with a chiral acid; isolation of a product of formula VII.chiral acid. Optionally, the undesired isomers in the mother liquor are racemized by treatment with acid or base, optionally at higher temperature, and the racemic morpholine derivative is resubjected to a crystallization with a chiral acid. Optionally, the racemization of the undesired isomers and the crystallization of the desired isomer as a chiral acid addition salt is performed in a one pot fashion;

Preferred protecting groups (R₁) for the amino alcohol of formula VI are benzyl or substituted benzyl. Most preferably, R₁ is benzyl. Instead of benzyl, other nitrogen protecting groups, which are known to the skilled person (see e.g. Theodora W. Greene, Peter G. M. Wuts, Protecting Groups in Organic Syntheses, 3^{rd} ed., 1999, John Wiley & Sons) can be used.

If nitrogen protecting groups other then benzyl or derivatives thereof are used, step c) from the invention has to be modified accordingly (for removal of such protecting groups see Theodora W. Greene, Peter G. M. Wuts, Protecting Groups in Organic Syntheses, 3^{rd} ed., 1999, John Wiley & Sons).

Preferably, R₂ and R₃ are the same or different and are independently chosen from hydrogen, substituted or unsubstituted C₁₋₆ alkyl, substituted or unsubstituted C₁₋₁₀ aralkyl, or form a ring —(CH₂)ₙ—, wherein n=1-5. Most preferably, R₂ and R₃ are hydrogen.

Preferred chiral acids are tartaric acid or tartaric acid derivatives such as di-O,O′-toluoyl tartaric acid, di-O,0,-benzoyl tartaric acid, di-O,O′-anisoyl tartaric acid, or O,O′-dibenzoyl tartaric acid mono(dimethylamide), camphorsulfonic acid derivatives such as 3-bromocamphor-10-sulfonic acid, camphanic acid, 10-camphorsulfonic, or camphoric acid, amino acids such as glutamic acid, valine, or aspartic acid, mandelic acid or mandelic acid derivatives such as α-methoxy-α-trifluoromethylphenylacetic or α-methoxyphenylacetic acid, acetoxy-5-etienic acid, malic acid, menthyloxyacetic acid, N-(α-methylbenzyl)succinamidic acid, N-[1-naphthyl)pethyl]succinamic acid, N-(1-phenylethyl)succinamic acid, 1-mono-menthyl phthalate, N,N-Bis[1-phenylethyl]phthalamic acid, N-(1-phenylethyl)phthalamic acid, 2-phenylpropionic acid, phenylcarbamoyloxypropionic acid, pyroglutamic acid, quinic acid, 1,4-benzodioxane-2-carboxylic acid, 1,1′-binaphthalene-2,2′-diyl hydrogen phosphate, or 5-oxo-2-tetrahydrofurancarboxylic acid in either enantiomeric or diastereomeric form. However, the invention is not limited to these chiral acids.

Most preferably, the chiral acid is di-O,O′-toluoyl-L-tartaric acid.

The amino alcohol VI, glyoxal (IV), and the phenylboronic acid V are dissolved in an organic solvent in the presence or absence of water or in a mixture of organic solvents with or without water. The order of addition is not critical. The reagents can be added in any order. In all cases, compound of formula VII in the form of its free base is formed.

Suitable organic solvents are e.g. alcohols, such as ethanol, n-butanol, sec-butanol, tert-butanol, methanol, 2-propanol, or toluene, tetrahydrofuran, acetonitrile, DMF, DMSO, dioxane, DME, diglyme, nitromethane, methyl tert-butyl ether, CH₂Cl₂, or NMP or mixtures thereof, with toluene and ethanol, 2-propanol, n-butanol, sec-butanol, or tert-butanol, 2-butanol being particularly preferred.

The three-component coupling of compound VI, glyoxal, and phenylboronic acid of formula V is performed with 0.5 to 1.5 equivalents of boronic acid derivative of formula V relative to amino alcohol of formula VI, more preferably with 0.9 to 1.1 equivalents and with 0.8 to 1.5 equivalents of glyoxal relative to amino alcohol of formula VI, more preferably with 1.1 to 1.3 equivalents.

The three-component coupling is performed at a temperature between −20° C. and 100° C., more preferably between 20° C. and 50° C., most preferably at 25° C.

The conversion of amino alcohol of formula VI to the product (compound VII) is greater than 50%, usually greater than 95%, more preferably greater than 98%.

The isolation of the product of formula VII from the reaction mixture can be performed according to methods known to a person skilled in the art. Such methods include extraction, distillation, crystallization, or chromatography.

In a preferred embodiment of the invention, compound VII is isolated as an addition salt with a chiral acid. By correct choice of the chiral acid, only one out of four diastereoisomers crystallizes. The solvent of this crystallization is same or different to the solvent of the three component coupling. The solvent in which the crystallization is carried out can be chosen from protic or aprotic solvents or mixtures thereof. Typical solvents are alcohols such as ethanol, 2-propanol, 2-butanol, or n-butanol. Optionally the alcohol can be mixed with water or an apolar solvent such as toluene or heptane. However, the invention is not limited to these combinations. In a preferred embodiment, the crystallization is carried out in an alcohol with or without cosolvent. Most preferably the crystallization is carried out in mixtures of 2-propanol and water.

The crystallization is started at elevated temperature and cooling is performed either gradually or using a cooling ramp. The temperature of the crystallization depends on the solvents in use. The start of the crystallization can be at reflux temperature or below.

In a preferred embodiment, the crystallization is carried out in an alcohol and the initial temperature is between 30° C. and 100° C., more preferably, between 40° C. and 50° C. and the reaction mixture is gradually cooled to below 30° C., more preferably, to 0° C. to 10° C.

The crystallization can be carried out using 0.4 to 2.0 acid equivalents. This means that 0.4 to 2.0 equivalents of a carboxylic acid with one carboxyl group, 0.2 to 1.0 equivalents of a carboxylic acid with two carboxyl groups, etc. can be used.

A characteristic of the crystallization according to the invention is that a considerably larger amount of one enantiomer out of four possible diastereoisomers of the product VII crystallizes as addition salt with the chiral acid. Compound VII.chiral acid is typically obtained with an enantiomeric excess (ee) of >50%. In a preferred embodiment the ee is greater 90%. Enantiomeric excess refers to the ratio of diastereoisomers with 3-R configuration to diastereoisomers with 3-S configuration.

The optical purity of salt VII obtained after isolation may be improved before further processing. Improvement of the optical purity may be achieved e.g. by recrystallization.

The crystalline product VII.chiral acid is isolated by filtration.

Optionally, the mother liquor is heated to such a temperature that racemization of the undesired 3-epimer occurs. A preferred temperature for the racemization is 70-100° C. Cooling of the (now racemic) mixture leads to precipitation of the desired crystalline product (VII.chiral acid). The process can be repeated several times. Optionally, the reaction mass is concentrated between the individual crystallization/racemization cycles. Optionally an acid, such as HCl or H₂SO₄, is added which facilitates the razemisation.

In another embodiment of the invention, the mother liquor is treated with base and compound VII free base is extracted into an organic layer. Compound VII free base is then submitted to a racemization by addition of a base or addition of an acid or by stirring at elevated temperature or by a combination of two of these measures. Preferred bases are NaOH or other metal hydroxides. Racemic compound VII is then crystallized with a chiral acid as described above.

In another embodiment, crystallization and racemization are performed in a one pot fashion. This is done by carrying out the crystallization under conditions where the desired isomer cyrstallizes while the remaining isomers in the mother liquor undergo racemization;

b) generation of hemi-acetal VII free base by portioning compound of formula VII.chiral acid between an alkaline aqueous layer and a water-immiscible organic phase; activation of the hemi-acetal functionality by transforming the OH-group in an activated derivative; reaction of the activated acetal with alcohol of formula VIII. Removal of impurities by extraction to get a solution of compound IX;

The conversion of compound VII to aprepitant can be performed according to e.g. Zhao, M. M.; McNamara, J. M.; Ho, G.-J.; Emerson, K. M.; Song, Z. J.; Tschaen, D. M.; Brands, K. M. J.; Dolling, U.-H.; Grabowski, E. J. J.; Reider, P. J., J. Org. Chem. 2002, 67, 6743-6747 or WO2001096319 A1.

Surprisingly, we found that the isolation of certain intermediates can be omitted without loss in quality of aprepitant. In particular the isolation of IX, X, and III can be omitted, thereby resulting in a highly economical process. Most preferably, the synthesis of aprepitant starting from VII free base is carried out by isolating only compound XI. In another preferred embodiment of the invention, only intermediate III or intermediate X is isolated en route to aprepitant starting from VII free base. In other embodiments of the invention, other intermediates are additionally isolated.

For the conversion of VII.chiral acid to VII free base, VII.chiral acid is suspended in a mixture of water and of a water-immiscible organic solvent. Addition of base, preferably aqueous NaOH, NaHCO₃, or Na₂CO₃ generates compound VII free base which is extracted into the organic layer.

The organic layer can be dried by e.g. azeotropic distillation or addition of a drying agent, which is removed prior to further processing. Compound VII free base is then activated for coupling with alcohol VIII. In a preferred embodiment, the activation is carried out by treatment of VII free base with a base, preferably K₂CO₃, and Cl₃CCN, or a base and trifluoroacetic acid anhydride. Other activation methods which are known to a person skilled in the art can be applied. After activation, coupling is performed with 1.0 to 2.0 equivalents of alcohol VIII. Activation and coupling of compound VII free base or diastereoisomers and derivatives thereof with alcohol VIII are described in Zhao, M. M.; McNamara, J. M.; Ho, G.-J.; Emerson, K. M.; Song, Z. J.; Tschaen, D. M.; Brands, K. M. J.; Dolling, U.-H.; Grabowski, E. J. J.; Reider, P. J., J. Org. Chem. 2002, 67, 6743-6747. The conditions used in this publication can be applied to the described process.

In a preferred embodiment compound IX is further processed in solution after work-up without isolation. Work-up includes washing of the reaction mass with aqueous base, or aqueous acid, or both consecutively and in any order. Optionally, compound IX is isolated by crystallization;

c) removal of the N-protecting group to give the amine of formula X;

The removal of the protecting group depends on the nature of the protecting group and can be performed with methods known to a person skilled in the art. Such protecting groups and methods for their removal are described in Theodora W. Greene, Peter G. M. Wuts, Protecting Groups in Organic Syntheses, 3^{rd} ed., 1999, John Wiley & Sons.

In a preferred embodiment of the invention, R₁=benzyl or substituted benzyl and the protecting group is removed by hydrogenolysis. Such a conversion is described in Zhao, M. M.; McNamara, J. M.; Ho, G.-J.; Emerson, K. M.; Song, Z. J.; Tschaen, D. M.; Brands, K. M. J.; Dolling, U.-H.; Grabowski, E. J. J.; Reider, P. J., J. Org. Chem. 2002, 67, 6743-6747. If R₁=benzyl, the protecting group is removed by hydrogenolysis using H₂ or a hydrogen donor in the presence of a catalyst such as Pd.

After removal of the protecting group a compound of formula X is obtained. In a preferred embodiment, compound X is further processed in solution after work-up without isolation. Optionally, compound X is isolated by crystallization. Work-up includes optionally filtration of a catalysts and washing of the reaction mass with aqueous base, or aqueous acid, or both consecutively and in any order;

d) oxidation of the amine of formula X to the corresponding cyclic imine of formula XI; isolation of the cyclic imine of formula XI;

Suitable systems for such oxidations are combinations of an oxidizing agent and a base. In a preferred embodiment, N-chlorosuccinimid or N-bromosuccinimid in combination with DBU are used as reagents and the reaction is carried out in DMF as solvent. Another preferred oxidation is based on the use of NaOCl as oxidizing agent.

After the reaction and aqueous work-up, the product of formula XI is isolated by crystallization. Preferably, compound XI is crystallized from an alcohol, or a mixture of an alcohol and water or an organic cosolvent. Imin XI is obtained in high purity; the level of diastereoisomer iso-XI is below 5%, preferentially below 1%.

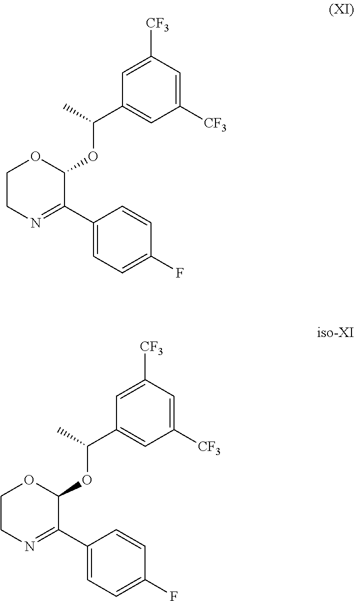

e) reduction of imine XI with a catalyst and H₂ or an H₂ equivalent; removal of the catalyst by filtration to get the key intermediate of formula III;

The reduction of the imine XI can be performed with H₂ in the presence of a catalyst such as Pd/C. Alternatively, a transfer hydrogenation using formates or other H₂-donors, which are known to the person skilled in the art, can be applied for the reduction of imine XI. Imine XI can also be reduced with complex hydrides such as NaBH₄ of LiAIH₄. In a preferred embodiment, imine XI is reduced with H₂ or a hydrogen donor such as potassium formate in the presence of Pd/C. The reduction of XI using Pd/C with molecular H₂ is described in Zhao, M. M.; McNamara, J. M.; Ho, G.-J.; Emerson, K. M.; Song, Z. J.; Tschaen, D. M.; Brands, K. M. J.; Dolling, U.-H.; Grabowski, E. J. J.; Reider, P. J., J. Org. Chem. 2002, 67, 6743-6747.

After the reduction, the catalyst is removed by filtration. In a preferred embodiment the filtrate is directly used in the next step.

Alternatively, the solvent can be removed and a solvent more suitable for the next step can be added;

f) alkylation of compound III to give aprepitant or fosaprepitant directly or via e.g. protected intermediates;

g) optionally, conversion of aprepitant to fosaprepitant by phosphorylation or a phosphorylation—deprotection sequence.

The alkylation is carried out using a solution of compound III according to prior art processes, which are described in EP 0734381 B1, WO 99065900 A1, WO 2001096315 A1, or WO 2003089429 A1.

Fosaprepitant is prepared from aprepitant or III as described in WO2006060110 A1 or EP 0748320 B1.

The present invention further relates to the following new compounds. These compounds are potential impurities in the process. A major advantage of the described process is that the each level of the compounds iso-XI, iso-I, and iso-II in aprepitant or fosaprepitant, respectively, is below 1%, preferably below 0.5%, most preferably below 0.1%.

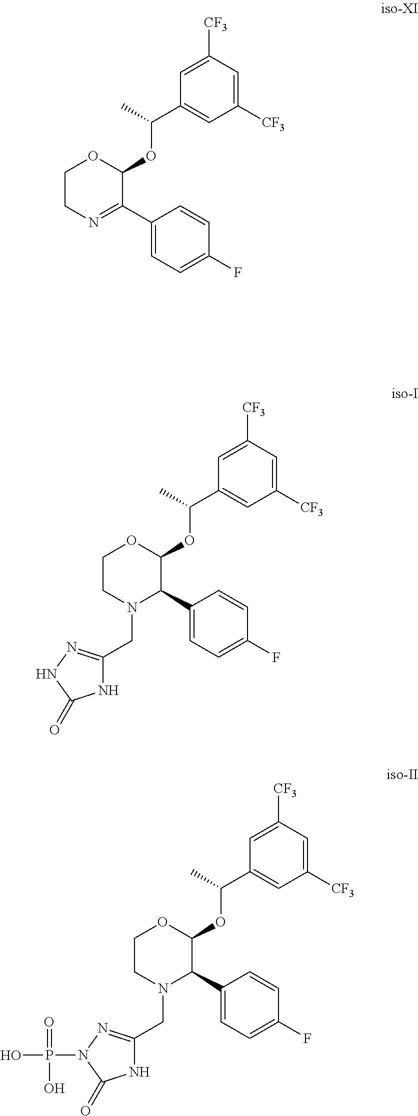

These compounds may be prepared directly via the same reaction sequence as used for the synthesis of aprepitant or fosaprepitant (steps a-g), if the compound of formula iso-VII is isolated as an addition salt with a chiral acid in step a and the compounds of formula iso-I or iso-II are obtained following the subsequent reaction steps via intermediate compound of formula iso-XI.

### EXAMPLES

The following examples describe the present invention in detail, but they are not to be construed to be in any way limiting for the present invention.

### Example 1

### Synthesis of VII.L-DTTA

In a 2 L reaction vessel equipped with a half-moon propeller and a thermomether 96.0 mL of a 40% aqueous glyoxal solution (1.2 equivalents, 840 mmol) were dissolved in 1400 mL of toluene. Then 100.8 mL of N-benzylaminoethanol (1 equivalent, 700 mmol) were added. During addition the temperature rises from 25° C. to 35° C. The resulting grey suspension was stirred at 35° C. for 30 minutes. In the meantime 102.8 g of ρ-fluor-phenyl-boronic acid (1.05 equivalents, 735 mmol) were dissolved in 500 mL of ethanol. A brown slightly turbid solution was formed. This solution was added to the reaction mixture within 45 minutes. The reaction was stirred at 35° C. for 1.5 h and then 1000 mL of water were added (pH of the resulting solution=5.8). Then 500 mL of saturated aqueous sodium hydrogen carbonate solution were added (pH=7.6) and the reaction mixture was stirred for 5 min at 25° C. The layers were separated and the organic layer was washed consecutively with 500 mL of a 4.3% aqueous sodium hydrogen carbonate solution and with 500 mL of a 13.3% aqueous sodium chloride solution. The main part of toluene was removed under reduced pressure (70° C., 100 mbar) to yield 234 g of a yellow oil. This oil was dissolved in 1169 g of isopropanol at 40° C. Then 149 g of L-DTTA (0.55 equivalents, 385 mmol) were added. The reaction mixture was heated to 55° C. and seeded. The resulting suspension was cooled to 22° C. and stirred over night. Then 1169 mL of water were added dropwise and the mixture was allowed to crystallize for 1 h at 22° C. Then the suspension was cooled to 0° C. and stirred for 17 h. The product is filtered off and washed twice with 200 mL of a cold (0° C.) mixture of isopropanol and water (1:1) to yield 221.1g of wet product (ee=78%). In a 3 L reaction vessel the crude material was dissolved in 1050 mL of isopropanol at 45° C. to give a clear solution. Then 1050 mL of water were added under stirring at this temperature to give a clear yellow solution which was cooled slowly to 38° C. and the product started to crystallize. The suspension was stirred for 45 min at 38° C. and was then cooled slowly to 0° C. and stirred for 30min. The resulting crystals were collected by filtration, washed with 500 mL of a mixture of isopropanol and water (1:1), and dried at 45° C. and 20 mbar to give 124 g of the title compound as white crystals (ee=99%).

### Example 1a

### Synthesis of VII.L-DTTA:

In a 2 L reaction vessel equipped with a half-moon propeller and a thermomether 96.0 mL of a 40% aqueous glyoxal solution (1.2 equivalents, 840 mmol) were dissolved in 1400 mL of toluene. Then 100.8 mL of N-benzylaminoethanol (1 equivalent, 700 mmol) were added. During addition the temperature rises from 25° C. to 29° C. The resulting grey suspension was stirred at 35° C. for 30 minutes. In the meantime 102.8 g of ρ-fluor-phenyl-boronic acid (1.05 equivalents, 735 mmol) were dissolved in 500 mL of ethanol. A brown slightly turbid solution was formed. This solution was added to the reaction mixture within 15 minutes. The reaction was stirred at 35° C. for 1 h (pH=5.26) and then 500 mL of a 4.3% aqueous sodium hydrogen carbonate solution were added (pH of the resulting solution =7.85). The reaction mixture was stirred for 5 min at 35° C. The layers were separated and the organic layer was washed consecutively with 500 mL of a 4.3% aqueous sodium hydrogen carbonate solution and with 500 mL of a 13.3% aqueous sodium chloride solution. The main part of toluene was removed under reduced pressure (60° C., 20 mbar) to yield 201 g of a yellow oil. This oil was dissolved in 1792 mL of isopropanol at 55° C. Then 149 g of L-DTTA (0.55 equivalents, 385 mmol) and 1407 mL of water were added. The reaction mixture was heated to 58° C. and the resulting clear solution was cooled to 22° C. and seeded. The suspension was stirred over night at 10° C. The product was filtered off and washed three times with 200 mL of a cold (0° C.) mixture of isopropanol and water (1:1) to yield 132 g of the desired product (ee=99%). The mother liquor was concentrated under reduced pressure (80 mbar) at 50° C. The aqueous residue was dissolved in dichloromethane and a 4.3% aqueous sodium hydrogen carbonate solution. A strong gas development was observed. The mixture was stirred for 15 min. The layers were separated and the organic layer was washed with a 4.3% aqueous sodium hydrogen carbonate solution. Then the dichloromethane layer was concentrated under reduced pressure (10 mbar) at 45° C. to give an orange-brown oil. The oil was dissolved in isopropanol and water (1:1). The reaction mixture was warmed to 45±5° C. and 0.5 equivalents of sodium hydroxide were added (pH 8±0.5→13±0.5). After 2 hours full racemization of the mixture was determined. The solution was neutralized with HCl. Then the reaction mixture was heated to 50° C. and 1.1 equivalents of L-DTTA and water were added. A clear solution was formed which was cooled to 20° C. and seeded. The reaction mixture was stirred over night at 10° C. The crystals were filtered off and washed 3 times with isopropanol/water (1:1). The product was dried under reduced pressure 20 mbar, 40° C. This racemization-crystallization procedure was repeated three times to give 367 g (78%) of the title compound (ee>99%).

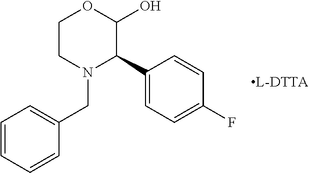

¹H-NMR (DMSO-d6, 300 MHz) δ (ppm)=2.18 (dt, CH₂, 1H, J 11.6 Hz, J 2.9 Hz), 2.40 (s, CH₃, 6H), 2.55 (m, CH₂, 1H), 2.92 (d, CH₂, 1H, J 13.4 Hz), 3.06 (d, CH, 1H, J 7.2 Hz), 3.52 (d, CH₂, 1H, J 13.4 Hz), 3.64 (t, CH₂, 1H, J 11.4 Hz), 3.79 (d, CH, 1H, J 10.7 Hz), 4.45 (d, CH, 1H, 7.0 Hz), 5.85 (s, CH, 2H), 7.23 (m, CH, 7H), 7.40 (d, CH, 2H, J 8.0 Hz), 7.51(t, CH, 2H, J 8.0 Hz), 7.92 (d, CH, 4H, J 8.0 Hz).

¹³C-NMR (DMSO-d6, 75.47 MHz) 6 (ppm)=22.12, 51.27, 58.92, 64.13, 72.23, 72.28, 98.24, 115. 64, 115.92, 126.67, 127.82, 129.06, 129.35, 130.34, 130.43, 131.37, 131.47, 136.92, 138.82, 145.45, 160.71, 163.93, 165.54, 168.15.

[α]_{D}²⁰=−77.6° (10 mg/1 mL acetonitrile)

MS: [VII+H]⁺ 288,0 (100%), [DTTA+H]⁺ 387,3 (20%), [DTTA+NH₄]⁺ 404,4 (15%), [DTTA+Na]³⁰ 409,3 (10%), [VII.DTTA+H]+ 674,3 (15%).

Mp: 150-152° C.

### Example 2

### Compound VII

20 g (29.7mmol) of VII.L-DTTA were dissolved in 200 mL of toluene before 200 mL of a 4.3% aqueous sodium hydrogen carbonate solution were added. The mixture was stirred at ambient temperature for 10 min. The layers were separated and the organic layer was dried with sodium sulfate. After filtration the organic layer was concentrated to give 8.4 g of the title compound which was used in the next step without further purification.

An analytical sample was purified by column chromatography on silica using pentane/ether as eluent.

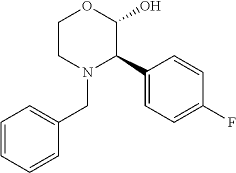

¹H-NMR (CDCl₂, 300 Hz) δ (ppm)=2.33 (dt, CH₂, 1H, J 11.6 Hz, J 3.6 Hz), 2.75 (td, CH₂, 1H, J 9.9Hz, J 2.0 Hz), 2.96 (d, CH₂, 1H, J 13.4 Hz), 3.16 (d, CH, 1H, J 7.2 Hz), 3.57 (bs, OH, 1H), 3.73 (d, CH₂, 1H, J 13.4 Hz), 3.39 (m, CH₂, 2H), 4.71 (d, CH, 1H, 7.1 Hz), 7.12 (t, CH, 2H, J 8.7 Hz), 7.30 (m, CH, 5H), 7.54 (m, CH, 2H). ¹³C-NMR (CDCl₃, 75.47 MHz) 6 (ppm)=50.88, 59.15, 64.83, 72.38, 98.13, 115.76, 116.04, 127.53, 128.71, 129.13, 130.78, 130.89, 131.08, 135.00, 138.58, 161.22, 164.48.

### Example 3

### Synthesis of Compound IX

a) Formation of the trichloroacetimidate

To a solution of VII free base (42.6 g, 148 mmol, 1 equivalent) in 150 mL of toluene were added 26.6 g of potassium carbonate (193 mmol, 1.3 equivalents) and 25.3 mL of trichloroacetonitrile (36.4 g, 252 mmol, 1.7 equivalents). The reaction mixture was stirred for 1 h at ambient temperature.

The suspension was filtered and the filtrate was concentrated to about 50% of the initial volume. The resulting solution was used in the next step without further purification

An analytical sample was prepared by complete evaporation of the solvent.

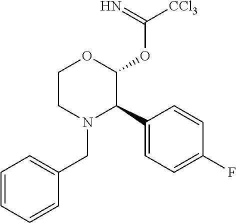

¹H-NMR (CDCl₃, 300 MHz) 6 (ppm)=1.43 (d, CH₃, 3H, J 6.6 Hz), 2.35 (dt, CH₂, 1H, J 11.7 Hz, J 3.1 Hz), 2.72 (d, CH₂, 1H, J 11.7 Hz), 2.92 (d, CH₂, 1H, J 13.4 Hz), 3.25 (d, CH, 1H, J 7.4 Hz), 3.68 (d, CH₂, 1H, J 13.4), 3.72 (m, CH₂, 1H), 3.97 (dd, CH₂, 1H, J 11.4 Hz, J 1.7 Hz), 4.20 (d, CH, 1H, J 7.4 Hz), 4.98 (q, CH, 1H, J 6.6 Hz), 7.02 (t, CH, 2H, J 8.6 Hz), 7.15-7.45 (m, CH aromatic, 10H), 7.71 (s, CH, 1H).

¹³C-NMR (CDCl₃, 75.47 MHz) 6 (ppm)=24.78, 51.12, 59.13, 64.92, 70.90, 74.25, 101.48, 115.69, 115.96, 121.98, 126.64, 127.46, 128.64, 129.05, 130.50, 131.74, 132.18, 134.52, 138.59, 145.75, 161.20, 164.46.

[α]_{D}²⁰=+25.2° C. (10 mg/1 mL acetonitrile)

b) Coupling

To the concentrated mixture of trichloroacetimidate from step a) were added 40.0 g (154 mmol, 1 equivalent) of chiral alcohol VIII. After stirring for 5 min at ambient temperature a clear solution was formed. The mixture was cooled to −10° C. and 2.94 mL (23 mmol, 0.15 equivalents) of BF₃—Et₂O were added dropwise at this temperature within 15 min. The reaction mixture was stirred for 45 min at −10° C. before 600 mL of a 4.3% aqueous sodium hydrogen carbonate solution was added. The layers were separated and the organic layer was washed twice with 500 mL of a 4.3% aqueous sodium hydrogen carbonate solution and was then concentrated under reduced pressure (60° C., 40 mbar) to yield 91.6 g (98%) of the title compound which was used in the next step without further purification.

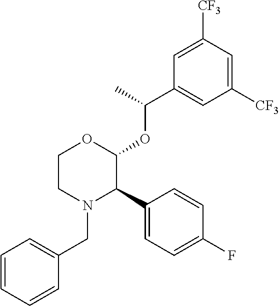

¹H-NMR (CDCl₃, 300 MHz) δ (ppm)=2.49 (dt, CH₂, 1H, J 10.6 Hz, J 3.6 Hz), 2.88 (td, CH₂, 1H, J 12.0 Hz, J 2.5), 3.12 (d, CH₂, 1H, J 13.5 Hz), 3.61 (d, CH, 1H, J 7.2 Hz), 3.83 (d, CH₂, 1H, J 13.5 Hz), 4.02 (m, CH₂, 1H), 4.15 (m, CH₂, 1H), 5.92 (d, CH, 1H, J 7.2 Hz), 7.08-7.43 (m, CHaromatic, 9H), 8.50 (s, NH, 1H).

¹³C-NMR (CDCl₃, 75.47 MHz) δ (ppm)=50.25, 58.90, 65.46, 68.94, 91.20, 99.24, 115.71, 115.99, 127.67, 128.89, 129.52, 131.20, 131.31, 133.21, 138.36, 161.36, 161.46, 164.67.

### Example 4

### Synthesis of compound X

108.9 g (206.4 mmol, 1 equivalent) of IX were dissolved in 360 mL of methanol in a 2 L round bottom flask. Then 78,5 g (412.8 mmol, 2 equivalents) of para-toluene sulfonic acid and 21.8 g of Pd/C (10%) were added. The reaction vessel was charged with hydrogen and the mixture was stirred for 16 h at ambient temperature. The catalyst was filtered off and washed three times with 50 mL of methanol. The filtrate was concentrated under reduced pressure (45° C., 10 mbar) to yield a suspension which was dissolved in 620 mL of dichloromethane and 500 mL of a 4.3% aqueous sodium hydrogen carbonate solution. The layers were separated and the organic layer was washed with 250 mL portions of a 4.3% aqueous sodium hydrogen carbonate solution until complete depletetion of the sulfonic acid was detected by HPLC. The organic layer was concentrated under reduced pressure (45° C., 10 mbar) to give 70.8 g (78%) of the title compound which was used in the next step without further purification.

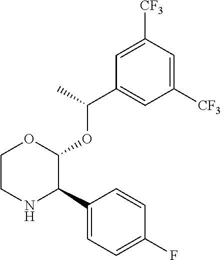

¹H-NMR (DMSO-d6, 300 MHz) δ (ppm)=1.29 (d, CH₃, 3H, J 6.6 Hz), 2.79 (m, CH₂, 2H), 3.55 (d, CH, 1H, J 7.4), 3.61 (dt, CH₂, 1H, J 11.0 Hz, J 2.6 Hz), 3.90 (d, CH₂, 1H, J 10.9 Hz), 4.07 (d, CH, 1H, J 7.4 Hz), 5.07 (q, CH, 1H, J 6.6 Hz), 7.09 (t, CH, 2H, J 8.8 Hz), 7.34 (m, CH, 2H), 7.39 (s, CH, 2H), 7.76 (s, CH, 1H).

¹³C-NMR (DMSO-d6, 75.47 MHz) δ (ppm)=24.63, 45.34, 63.66, 66.04, 73.01, 101.84, 114.60, 114.88, 117.95, 120.94, 120.99, 121.04, 121.57, 125.18, 126.59, 128.79, 129.86, 129.96, 130.36, 130.79, 131.23, 136.48, 136.52, 146.94, 160.40, 163.63.

### Example 5

### Synthesis of XI

25.0 g (57.1 mmol, 1 equivalent) of X were dissolved in 39.5 mL of dimethylformamide and 1.58 g (11.4 mmol, 0.2 equivalents) of potassium carbonate were added. Then the suspension was cooled to −5° C. before 8.78 g (65.7 mmol, 1.2 equivalents) of NCS were added within 15 minutes. The reaction mixture was stirred at −5° C. for 5 min before 10.7 mL (71.5 mmol, 1.3 equivalents) of DBU were added within 20 min. The reaction mixture was stirred at −5° C. for 5 h. Then 102 mL of water and 51 mL of dichloromethane were added and the layers were separated. The organic layer was washed 5 times with 100 mL of a 4% aqueous lithium chloride solution and concentrated under reduced pressure (45° C., 10 mbar) to yield 21.8 g of an orange suspension. The suspension was dissolved at 47° C. in 68.2 mL of isopropanol. The mixture was cooled slowly to ambient temperature, while crystallization starts. The suspension was stirred for 45 min before 17.8 mL of water were added. The suspension was then cooled to 0° C. and stirred for 1.5 h. The crystals were collected by filtration and washed three times with 20 mL of a cold mixture of isopropanol/water (3.8:1.0) to yield 16.9 g (78%) of the title compound as a white crystalline product after drying at 40° C. under reduced pressure.

### Example 5a

### Synthesis of XI

1.0 g (2.29 mmol, 1 equivalent) of X were dissolved in 1.58 mL of acetonitrile and 63 mg (0.46 mmol, 0.2 equivalents) of potassium carbonate were added, before 8.16 mL (13.7 mmol, 6.0 equivalents) of NaOCl (10%) were added within 15 minutes. Then 2.22 mL (14.9 mmol, 6.5 equivalents) of DBU were added. The reaction mixture was stirred at ambient temperature for 14 h. Then 50 mL of water and 50 mL of dichloromethane were added and the layers were separated and concentrated under reduced pressure (45° C., 10 mbar) to yield 0.92 g (92%) of XI.

### Example 6

### Synthesis of XI Without Isolation of Intermediates

In a 2 L reaction vessel equipped with a half-moon propeller and a thermomether 96.0 mL of a 40% aqueous glyoxal solution (1.2 equivalents, 840 mmol) were dissolved in 1400 mL of toluene. Then 100.8 mL of N-benzylaminoethanol (1 equivalent, 700 mmol) were added. During addition the temperature rises from 25° C. to 35° C. The resulting grey suspension was stirred at 35° C. for 30 minutes. In the meantime 102.8 g of ρ-fluor-phenyl-boronic acid (1.05 equivalents, 735 mmol) were dissolved in 500 mL of ethanol giving a brown slightly turbid solution. This solution was added to the reaction mixture within 45 minutes. The reaction stirred at 35° C. for 1.5 h and then 500 mL of a 8.6% aqueous sodium hydrogen carbonate solution were added to the reaction mixture (pH=7.7) and the resulting mixture was stirred for 5 min at 25° C. The layers were separated and the organic layer was washed consecutively with 500 mL of a 4.3% aqueous sodium hydrogen carbonate solution and with 500 mL of a 13.3% aqueous sodium chloride solution. The combined aqueous layers were reextracted with 200 mL of toluene. The main part of toluene was removed under reduced pressure (70° C., 100 mbar) to yield 240 g of a yellow oil. The oil was dissolved in 1407 g of isopropanol at 55° C. Then 148.7 g of L-DTTA (0.55 equivalents, 385 mmol) were added. The reaction mixture was heated to 58° C. Then 1407 mL of water were added dropwise. Then the reaction mixture was allowed to crystallize for 1 h at 58° C. and then the suspension was cooled to 10° C. and stirred for 12 h. The product was filtered off and washed three times with 200 mL of a cold (0° C.) mixture of isopropanol and water (1:1) to yield 210 g of wet product which was dried at 45° C. and 20 mbar to give 161 g of VII.L-DTTA as white crystals (ee=98%).

VII.L-DTTA was dissolved in 900 mL of toluene before 2000 mL of a half saturated aqueous sodium hydrogen carbonate solution were added. The mixture was stirred at ambient temperature for 10 min. The layers were separated and the organic layer was concentrated to a mass of 69.5 g. The resulting residue was dissolved in 487 g of toluene and 43.5 g of potassium carbonate (315 mmol, 1.3 equivalents) and 41.2 mL of trichloroacetonitrile (411 mmol, 1.7 equivalents) were added. The reaction mixture was stirred for 55 min at ambient temperature. The suspension was filtered and the filtrate was concentrated to about 50% of the initial volume.

To the resulting residue 62.5 g (242 mmol, equivalent) of VIII were added. After stirring for 5 min at ambient temperature a clear solution was formed. The mixture was cooled to −10° C. and 4.60 mL (36 mmol, 0.15 equivalents) of BF₃—Et₂O were added dropwise at this temperature within 25 min. The reaction mixture was stirred at −10° C. for 20 min before 600 mL of a 4.3% aqueous sodium hydrogen carbonate solution were added. The mixture was allowed to come to ambient temperature and the layers were separated. The organic layer was washed twice with 500 mL of a 4.3% aqueous sodium hydrogen carbonate solution and concentrated under reduced pressure (60° C., 40 mbar) to yield 118.9 g of IX which were dissolved in 396 mL of methanol in a 2 L round bottom flask. Then 48.5 g (254.6 mmol, 2 equivalents) of para-toluene sulfonic acid and 13.4 g of Pd/C (10%) were added. The reaction vessel was charged with hydrogen and the reaction mixture was stirred for 5 h. The catalyst was filtered off and washed three times with 50 mL of methanole. The filtrate was concentrated under reduced pressure (45° C., 10 mbar) to yield 153 g of slightly red crystals. Then 500 mL of dichloromethane were added and the organic layer was neutralized with 800 mL of a 4.3% aqueous sodium hydrogen carbonate solution. The layers were separated and the organic layer was washed twice with 500 mL of a 4.3% aqueous sodium hydrogen carbonate solution and was then concentrated under reduced pressure (45° C., 10 mbar) to give 88 g of crude X.

The resulting crude X was dissolved in 109 mL of dimethylformamide and 4.36 g (31.6 mmol, 0.2 equivalents) of potassium carbonate were added. Then the suspension was cooled to −11° C. before 24.3 g (65.7 mmol, 1.2 equivalents) of NCS were added within 15 minutes. The reaction mixture was stirred at that temperature for 5min before 29.5 mL (197 mmol, 1.3 equivalents) of DBU were added at −5° C. in 25 min. The reaction mixture was stirred for 2 h at −5° C. and then 280 mL of water and 140 mL of dichloromethane were added and the layers were separated. The organic layer was washed 5 times with 280 mL of a 4% aqueous lithium chloride solution and concentrated under reduced pressure (45° C., 10 mbar) to yield in 73.9 g of an orange oil. The oil was dissolved at 47° C. in 96 mL isopropanol. The mixture was cooled slowly to ambient temperature, while crystallization starts. The suspension was stirred for 1 h at 0° C. The crystals were collected by filtration and washed three times with 50 mL of a cold mixture of isopropanol/water (3.8:1.0) to give 34.2 g (50%) of the title compound after drying at 40° C. under reduced pressure.

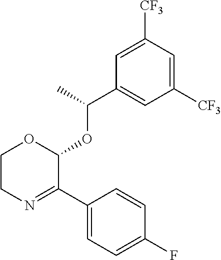

¹H-NMR (CDCl₃, 300 MHz) δ (ppm)=1.58 (d, CH₃, 3H, J 6.6 Hz), 3.84 (m, CH₂, 1H), 3.92 (m, CH₂, 2H), 4.10 (m, CH₂, 1H), 5.13 (s, CH, 1H), 5.16 (q, CH, 1H, J 6.6 Hz), 7.00 (t, CH, 2H, J 8.6 Hz), 7.46 (dt, CH, 2H, J 5.5 Hz, J 3.2 Hz), 7.79 (s, CH, 2H), 7.90 (s, CH, 1H). ¹³C-NMR (CDCl₃, 75.47 MHz) δ (ppm)=24.35, 48.35, 56.31, 73.51, 88.78, 115.59, 115.88, 121.79, 122.48, 122.53, 122.58, 125.41, 127.61, 128.77, 128.88, 131.74, 132.19, 132.63, 133.04, 145.24, 161.98, 162.81, 166.12.

[α]_{D}²⁰=+12.7° (10 mg/1 mL acetonitrile)

Crystallographic data:

Data collection has been performed on an Oxford Diffraction Gemini single crystal diffractometer. A single crystal of XI (0.01×0.03×0.12 mm in size) was studied at 173(2) K using graphite monochromatized Cu—K_{□}radiation. Basic crystallographic data are as follows: monoclinic symmetry, space group P12₁1, a=5.5081(1)Å, b=10.5758(2)Å, c=16.2303(4)Å, β=90.977(2)°, V=945.4(1)Å³, chemical formula C₂₀H₁₆F₇NO₂, Z=2. The structure was solved by direct methods and refined to a residual of R(|F|)=0.048 for 2237 independent observed reflections (I>2σ(I)) and 273 parameters.

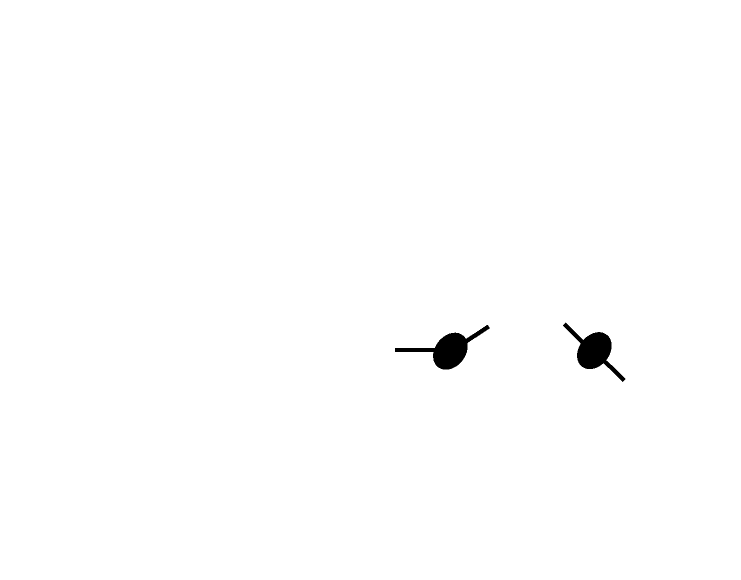

### Example 7

### Synthesis of III

22.2 g (51 mmol, 1 equivalent) of XI were dissolved in 330 mL of methanol. Then 2.78g of Pd/C (10%) were added. The flask was charged with hydrogen and the resulting mixture was stirred for 2 h at ambient temperature. The catalyst was filtered off and washed three times with 50 mL of methanol. The filtrate was concentrated under reduced pressure (45° C., 10 mbar) to yield 12.4 g (55.5%) of the title compound as a colourless oil which was used without further purification in the next step.

### Example 7a

### Synthesis of III

100 mg (0.23 mmol, 1 equivalent) of XI were dissolved in 1.3 mL of ethanol. Meanwhile 56 mg (0.67 mmol, 2.9 equivalents) of potassium formate were dissolved in 0.09 mL of water and 21 mg of Pd/C (20%) were added. Then the solution of XI in ethanol was added. The resulting mixture was stirred for 2 h at ambient temperature. The catalyst was filtered off and washed three times with 1 mL of methanol. The filtrate was concentrated under reduced pressure (45° C., 10 mbar) to yield 86 mg (86%) of the title compound as a colourless oil.

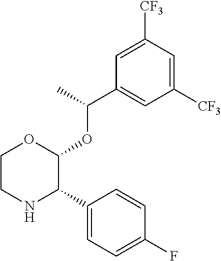

¹H-NMR (DMSO-d₆, 300 MHz) δ (ppm)=1.36 (d, CH₃, 3H, J 6.6 Hz), 2.97 (m, CH₂, 2H), 3.50 (d, CH, 1H, J 7.42), 3.92 (d, CH, 1H, J 2.3 Hz), 3.99 (m, CH₂, 1H), 4.42 (d, CH, 1H, J 2.3 Hz), 4.97 (q, CH, 1H, J 6.6 Hz), 7.03 (t, CH, 2H, J 8.4 Hz), 7.35 (dt, CH, 2H, J 5.8 Hz, J 2.8 Hz), 7.39 (s, CH, 2H), 7.80 (s, CH, 1H).

¹³C-NMR (DMSO-d₆, 75.47 MHz) δ (ppm)=24.68, 45.76, 59.21, 61.36, 71.82, 95.69, 114.52, 114.80, 121.13, 122.61, 125.23, 126.71, 128.84, 129.11, 129.21, 129.86, 130.29, 130.73, 131.16, 136.39, 136.43, 147.27, 160.06, 163.28.

### Example 8

### Synthesis of I

5-Chloromethyl-2,4-dihydro-[1,2,4]triazol-3-one-variant:

To a solution of 0.60 g (1.48 mmol, 1 equivalent) of III in 3.1 mL of DMF were added 226 mg (1.64 mmol, 1.1 equivalents) of potassium carbonate at ambient temperature. The mixture was stirred at 20° C. and a solution of 238 mg (1.78 mmol, 1.2 equivalents) of 5-Chloromethyl-2,4-dihydro-[1,2,4]triazol-3-one in 1.5 mL of DMF was added dropwise within 15 min. The reaction was stirred for 15 min at 20° C. before 10 mL of water were added dropwise while the product started to crystallize. The resulting suspension was stirred for 10 min at 25° C. before it is cooled to 0° C. and stirred for 1 h. The crystals was collected by filtration and washed with cold water to give 416 mg (68%) of the title compound after drying under reduced pressure (40° C., 10 mbar) as a white crystalline product.

### Example 8a

### Synthesis of I

N-[1-Amino-2-chloro-eth-(Z)-ylidene]-hydrazinecarboxylic acid methyl ester-variant:

To a solution of 500 mg (1.14 mmol, 1 equivalent) of III in 4.2 mL of acetonitrile were added 546 μL (3.29 mmol, 2.9 equivalents) of N,N-diisopropylethylamine and 244 mg (1.48 mmol, 1.29 equivalents) of N-[1-Amino-2-chloro-eth-(Z)-ylidene]-hydrazinecarboxylic acid methyl ester. The resulting suspension was stirred at ambient temperature for 3 h while a clear solution was formed. The reaction mixture was concentrated under reduced pressure (45° C., 100 mbar) and the residue was dissolved in 10 mL of dichloromethane and washed with 10 mL of a 26.5% aqueous sodium chloride solution. The organic layer was concentrated under reduced pressure (45° C., 100 mbar). Then 4.2 mL of acetonitrile were added to the residue and the mixture was transferred to a reactor where it was stirred for 55 h at 110° C. and 1.5 bar. Then the reaction mixture was concentrated under reduced pressure (45° C., 10 mbar) and the residue was dissolved in 5.6 mL of methanol. The reaction mixture was heated to reflux and charcoal was added. The reaction mixture was kept at reflux for 30 min before it was filtered over a bed of celite and washed with methanol. The filtrate was concentrated under reduced pressure and then suspended in acetonitrile. The resulting crystalline product I was collected by filtration and washed with cold acetonitrile to give 324 mg (53%) of the title compound as a white crystalline product.

### Example 9

### Synthesis of I Without Isolation of Intermediates

A mixture of 33.5 g (77 mmol, 1 equivalent) of XI dissolved in 496 mL of methanol and 6.69 g of Pd/C (10%) was charged with hydrogen and stirred for 2h at ambient temperature. The catalyst was filtered off and washed three times with 50 mL of methanol. The filtrate was concentrated under reduced pressure (45° C., 10 mbar) to yield in 34 g of III as a colourless oil. This oil was dissolved in 278 mL of acetonitrile and 28 g (218 mmol, 2.9 equivalents) of N,N-diisopropylethylamine and 16 g (97.8 mmol, 1.3 equivalents) of N-[1-Amino-2-chloro-eth-(Z)-ylidene]-hydrazinecarboxylic acid methyl ester were added. The mixture was stirred at ambient temperature for three hours and then concentrated under reduced pressure. The residue was dissolved in 300 mL of dichloromethane and washed with 300 mL of a 26.5% aqueous sodium chloride solution. The organic layer was concentrated under reduced pressure (45° C., 10 mbar). Then 130 mL acetonitrile were added and the resulting mixture was transferred to a reactor where it was stirred for 45 h at 110° C. and 1.5 bar. The reaction mixture was then concentrated under reduced pressure (45° C., 10 mbar) and the residue was dissolved in 371 mL methanol. The reaction mixture was heated to reflux and charcoal was added. The reaction mixture was kept at reflux for 30 min before it was filtered over a bed of celite and washed with methanol. The filtrate was concentrated under reduced pressure and then suspended in 278 mL of acetonitrile. The resulting crystals were collected by filtration and washed with acetonitrile to give 377 g (69%) of the title compound as a white crystalline product.

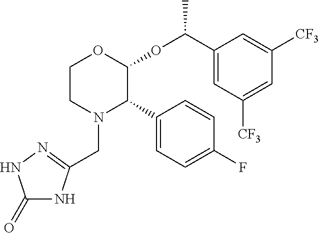

NMR: ¹H-NMR (DMSO-d₆, 300 MHz) δ (ppm)=1.36 (d, CH₃, 3H, J 6.5 Hz), 2.39 (dt, CH₂, 1H, J 11.7 Hz, J 3.1 Hz), 2.75 (d, CH₂, 1H, J 14.2 Hz), 2.84 (d, CH₂, 1H, J 11.7 Hz), 3.38 (d, CH₂, 1H, J 13.9 Hz), 3.49 (d, CH, 1H, J 2.54), 3.62 (d, CH₂, 1H, J 10.9 Hz), 4.12 (t, CH₂, 1H, J 9.9 Hz), 4.33 (d, CH, 1H, J 2.7 Hz), 4.94 (q, CH, 1H, J 6.5 Hz), 7.07 (t, CH, 2H, J 8.8 Hz), 7.37 (s, CH, 2H), 7.51 (t, CH, 2H, J 6.1 Hz), 7.83 (s, CH, 1H), 11.29 (bs, NH, 2H).

¹³C-NMR (DMSO-d₆, 75.47 MHz) δ (ppm)=24.75, 50.79, 51.88, 59.07, 68.02, 71.87, 95.77, 114.79, 115.07, 121.39, 121.60, 125.22, 126.87, 128.83, 129.88, 130.31, 130.74, 131.18, 131.37, 131.47, 133.52, 133.56, 144.24, 146.87, 156.72, 160.45, 163.68.

### Example 10

### Synthesis of iso-VII.D-DTTA

In a 2 L reaction vessel equipped with a half-moon propeller and a thermomether 96.0 mL of a 40% aqueous glyoxal solution (1.2 equivalents, 840 mmol) were dissolved in 1400 mL of toluene. Then 100.8 mL of N-benzylaminoethanol (1 equivalent, 700 mmol) were added. During addition the temperature rises from 25° C. to 35° C. The resulting grey suspension was stirred at 35° C. for 30 minutes. In the meantime 102.8 g of ρ-fluor-phenyl-boronic acid (1.05 equivalents, 735 mmol) were dissolved in 500 mL of ethanol. A brown slightly turbid solution was formed. This solution was added to the reaction mixture within 45 minutes. The reaction was stirred at 35° C. for 1.5 h and then 1000 mL of water were added (pH of the resulting solution=5.8). Then 500 mL of saturated aqueous sodium hydrogen carbonate solution were added (pH=7.6) and the reaction mixture was stirred for 5 min at 25° C. The layers were separated and the organic layer was washed consecutively with 500 mL of a 4.3% aqueous sodium hydrogen carbonate solution and with 500 mL of a 13.3% aqueous sodium chloride solution. The main part of toluene was removed under reduced pressure (70° C., 100 mbar) to yield 234 g of a yellow oil. This oil was dissolved in 1169 g of isopropanol at 40° C. Then 149 g of D-DTTA (0.55 equivalents, 385 mmol) were added. The reaction mixture was heated to 45° C. and 1620 mL of water were added dropwise at that temperature. The resulting mixture was cooled to 10° C. while crystallization started. The resulting suspension was stirred for 1 h at 10° C. and the crystals were filtered off and washed three times with 330 mL of a cold (0° C.) mixture of isopropanol and water (1:1) to yield 115 g of wet product (ee=87%). In a 2 L reaction vessel the crude material was dissolved in 575 mL of isopropanol and 6.3 g (0.1 equivalents) of D-DTTA were added and the mixture was heated to 50° C. Then 575 mL of water were added under stirring at 45° C. to give a clear yellow solution which was slowly cooled to 20° C. while crystallization starts. The resulting suspension was stirred for 45 min at 20° C. and was then cooled slowly to 10° C. and stirred for 1 h. The resulting crystals were collected by filtration, washed with 500 mL of a mixture of isopropanol and water (1:1), and dried at 45° C. and 20 mbar to give 72.8 g (68.6%) of the title compound as white crystals (ee=99.9%).

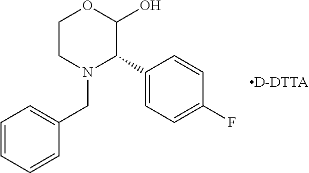

¹H-NMR (CDCl₃, 300 MHz) δ (ppm)=2.18 (dt, CH₂, 1H, J 11.4 Hz, J 2.6 Hz), 2.38 (s, CH₃, 6H), 2.58 (d, CH₂, 1H, J 11.7 Hz), 2.92 (d, CH₂, 1H, J 13.4 Hz), 3.07 (d, CH, 1H, J 7.2 Hz), 3.52 (d, CH₂, 1H, J 13.4 Hz), 3.64 (t, CH₂, 1H, J 11.5 Hz), 3.79 (d, CH, 1H, J 10.2 Hz), 4.46 (d, CH, 1H, 7.2 Hz), 5.85 (s, CH, 2H), 7.23 (m, CH, 7H), 7.39 (d, CH, 2H, J 8.0 Hz), 7.51(dt, CH, 2H, J 6.0 Hz, J 2.0), 7.93 (d, CH, 4H, J 8.0 Hz).

¹³C-NMR (CDCl₃, 75.47 MHz) δ (ppm)=21.60, 50.77, 58.43, 63.62, 71.78, 97.73, 115. 14, 115.42, 126.19, 127.33, 127.59, 128.56, 128.86, 129.34, 129.84, 129.91, 130.88, 130.98, 131.68, 131.79, 136.34, 136.38, 138.27, 144.93, 160.22, 163.44, 165.07, 167.68.

### Example 11

### Iso-IX

Iso-VII free base was prepared from iso-VII.D-DTTA. For this purpose 68 g (100.9 mmol) of D-DTTA are dissolved in 150 mL of toluene before 450 mL of a 4.3% aqueous sodium hydrogen carbonate solution are added (pH=7.1). The mixture is stirred at ambient temperature for 15 min, while CO₂ is formed. The layers are separated and the organic layer is washed two times with 150 mL of 2.9% aqueous sodium hydrogen carbonate solution and once with 100 mL of 13.3% saturated aqueous sodium chloride solution. The organic layer is concentrated to 28.6 g.

It is also possible to isolate iso-VII free base by removing toluene under reduced pressure. Then the product can be crystallized by adding 550 mL of n-heptane. Then the mixture is warmed up to 93° C. and a clear solution is formed. Then the reaction is allowed to cool down again, the crystallization starts at 71° C. The suspension is stirred for 60 min at 35° C. and then for 30 min at 10° C. The product is filtered off and washed two times with 150 mL of cold n-heptane. The product is dried under reduced pressure at 45° C. to yield in 22.8 g (78.6%, de: >99.9%) of the white product iso-VII.

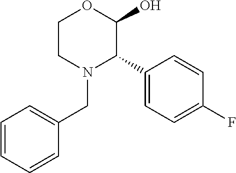

¹H-NMR (CDCl₃, 300 MHz) δ (ppm)=2.33 (dt, CH₂, 1H, J 11.6 Hz, J 3.6 Hz), 2.75 (d, CH₂, 1H, J 11.3 Hz), 2.97 (d, CH₂, 1H, J 13.4 Hz), 3.16 (d, CH, 1H, J 7.2 Hz), 3.74 (d, CH₂, 1H, J 13.4 Hz), 3.89 (m, CH₂, 2H), 4.70 (d, CH, 1H, 6.8 Hz), 7.13 (t, CH, 2H, J 8.7 Hz), 7.32 (m, CH, 5H), 7.54 (m, CH, 2H).

¹³C-NMR (CDCl₃, 75.47 MHz) δ (ppm)=50.92, 59.27, 64.87, 72.33, 98.14, 115.75, 116.03, 127.56, 128.73, 129.18, 130.82, 130.89, 131.06, 135.10, 138.61, 161.21, 164.47.

### Example 12

### Activated Iso-VII

To iso-VII free base (10.0 g 36 mmol 1 equivalent in 45 mL toluene) 6.45 g of potassium carbonate (47 mmol, 1.3 equivalents) and 6.12 mL of trichloroacetonitrile (61 mmol, 1.7 equivalents) are added at 35° C. The reaction mixture is stirred for 3.5 h at 35° C.

Then potassium carbonate is filtered off and washed two times with 10 mL toluene. The mixture is concentrated to about 50% of the initial volume. Optionally, toluene is removed completely. The residue corresponds to the structure of activated iso-VII.

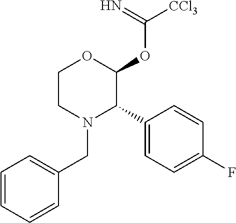

¹H-NMR (CDCl₃, 300 MHz) δ (ppm)=2.32 (dt, CH₂, 1H, J 10.6 Hz, J 3.5 Hz), 2.71 (td, CH₂, 1H, J 12.0 Hz, J 2.4), 2.95 (d, CH₂, 1H, J 13.5 Hz), 3.44 (d, CH, 1H, J 7.2 Hz), 3.66 (d, CH₂, 1H, J 13.5 Hz), 3.86 (dt, CH₂, 1H, J 10.7 Hz, J 2.5 Hz), 3.98 (m, CH₂, 1H), 5.54 (d, CH, 1H, J 7.2 Hz), 6.94 (t, CH, 2H, J 8.7 Hz), 7.18 (m, CH, 5H), 7.44(m, CH, 2H), 8.32 (s, NH, 1H).

¹³C-NMR (CDCl₃, 75.47 MHz) δ (ppm)=50.22, 58.86, 65.33, 68.91, 91.20, 99.21, 115.68, 115.91, 127.50, 128.79, 129.12, 131.17, 131.28, 133.18, 138.33, 161.34, 161.37, 164.63.

### Example 13

### Iso-IX

To the concentrated mixture of trichloroacetimidate from example 12 tetrahydrofuran (10 ml) and 9.27 g (36 mmol, 1 equivalent) of VIII are added. After stirring for 5 min. at ambient temperature a clear solution is formed. The mixture is cooled down to −5° C., and 1.36 mL (11 mmol, 0.3 equivalents) BF₃*Et₂O are added dropwise at this temperature within 15 min. The reaction mixture is stirred at this temperature for 45 min., before a 4.3% aqueous sodium hydrogen carbonate solution is added. The layers are separated and the organic layer is washed with 100 mL of an 26.5% aqueous sodium chloride solution. The product layer is concentrated under reduced pressure (60° C., 50 mbar) to yield in 17.22 g (91%) of iso-IX, which is transferred without any purification to iso-X.

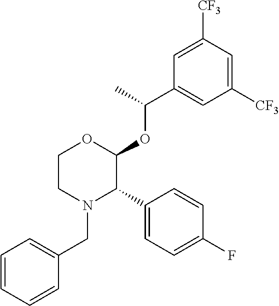

hu 1H-NMR (CDCl₃, 300 MHz) δ (ppm)=0.89 (d, CH₃, 3H, J 6.5 Hz), 2.23 (dt, CH₂, 1H, J 11.8 Hz, J 3.4 Hz), 2.64 (d, CH₂, 1H, J 11.8 Hz), 2.88 (d, CH₂, 1H, J 13.4 Hz), 3.17 (d, CH, 1H, J 7.4 Hz), 3.64 (dt, CH₂, 1H, J 8.6 Hz, J 2.3 Hz), 3.68 (d, CH₂, 1H, J 13.4 Hz), 3.79 (m, CH2, 1H), 4.42 (d, CH, 1H, J 7.3 Hz), 4.59 (q, CH, 1H, J 6.4 Hz), 7.04 (t, CH, 2H, J 8.7 Hz), 7.19 (m, CH, 5H), 7.47 (m, CH, 2H), 7.60 (s, CH, 2H), 7.66 (s, CH, 1H).

¹³C-NMR (CDCl₃, 75.47 MHz) δ (ppm)=24.78, 51.12, 59.13, 64.92, 70.90, 74.25, 101.48, 115.69, 115.96, 121.98, 126.64, 127.46, 128.64, 129.05, 130.50, 131.74, 132.18, 134.52, 138.59, 145.75, 161.20, 164.46.

[α]_{D}²⁹=+25.2° (10 mg/1 mL acetonitrile)

### Example 14

### Synthesis of Iso-X

17.22 g (32.6 mmol, 1 equivalent) of iso-IX are dissolved in 70 mL of methanol in a 250 mL round bottom flask. Then 9.93 g (52 mmol, 1.6 equivalents) of para-toluene sulfonic acid and 2.8 g (Pd/C 10%) are added. The reaction mixture is hydrogenated in 2.5 h using a hydrogen balloon. The catalyst is filtered of and washed three times with 10 mL of methanol. The filtrate is concentrated under reduced pressure (50° C., 100 mbar) to yield an oil. Then 100 mL of dichloromethane are added and the organic layer is neutralized with 100 mL of a 4.3% aqueous sodium hydrogen carbonate solution. The layers were separated and the organic layer is washed once with 100 mL of a 4.3% aqueous sodium hydrogen carbonate solution and once with 50 mL of a 26.5% aqueous sodium chloride solution, before the product is concentrated under reduced pressure (45° C., 10 mbar). 15.13 g (>99%) of the crude product iso-X were isolated.

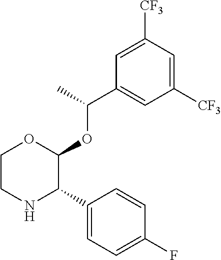

¹H-NMR (DMSO-d₆, 300 MHz) δ (ppm)=0.99 (d, CH₃, 3H, J 6.4 Hz), 2.78 (m, CH₂, 2H), 3.54 (d, CH, 1H, J 7.3), 3.73 (m, CH₂, 2H), 4.46 (d, CH, 1H, J 7.4 Hz), 4.90 (m, CH, 1H), 7.13 (t, CH, 2H, J 8.9 Hz), 7.48 (dt, CH, 2H, J 5.8 Hz, J 2.5 Hz), 7. 90 (s, CH, 2H), 8.01 (s, CH, 1H).

¹³C-NMR (DMSO-d₆, 75.47 MHz) δ (ppm)=21.96, 45.18, 63.61, 66.04, 67.34, 74.17, 102.50, 114.58, 115.35, 120.90, 120.95, 121.86, 125.47, 126.39, 126.80, 130.13, 130.18, 130.37, 130.56, 130.61, 136.95, 136.99, 147.90, 151.28, 160.23, 163.45.

### Example 15

### Synthesis Iso-XI

9.39 g (21.5 mmol, 1 equivalent) of iso-X are dissolved in 40 mL of dimethylformamide and 0.59 g (4.3 mmol, 0.2 equivalents) of potassium carbonate are added. Then the suspension is cooled down to −10° C., before 3.30 g (24.7 mmol, 1.2 equivalents) of NCS are added in 15 minutes. The reaction mixture is stirred at that temperature for 5 min more, before 10.7 mL (26.5 mmol, 1.3 equivalents) of DBU are added at −10° C. in 20 min. The reaction mixture is stirred for 30 min at this temperature. Then 102 mL of water and 51 mL of dichloromethane were added and the layers were separated. The organic layer is washed 5 times with 100 mL of a 4% aqueous lithium chloride solution. The organic layer is concentrated under reduced pressure (45° C., 10 mbar) to yield 9.4 g of an orange oil. The product is dissolved at 47° C. in 26.3 mL iso-propanol. The mixture is cooled down slowly to ambient temperature, while it starts crystallizing. The suspension is stirred for 45 min before 8.05 mL of water were added. The suspension is stirred for 1 h at 0° C. The product is filtered of and washed two times with 20 mL of a cold mixture of iso-propanol/water (3.8/1.0). The white crystalline product is dried at 40° C. under reduced pressure, to yield in 9.87 g (49%) of the product iso-XI.

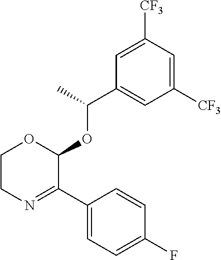

¹H-NMR (DMSO-d₆, 300 MHz) δ (ppm)=1.50 (d, CH₃, 3H, J 6.5 Hz), 3.36 (m, CH₂, 1H), 3.50 (m, CH₂, 1H), 3.70 (d, CH, 1H, J 3.8 Hz), 5.24 (q, CH, 1H, J 6.5 Hz), 5.80 (s, CH, 1H), 7.27 (t, CH, 2H, J 8.9 Hz), 7.92 (dt, CH, 2H, J 8.9 Hz, J 3.3Hz), 7.97 (s, CH, 1H), 8.03 (s, CH, 2H).

¹³C-NMR (DMSO-d₆, 75.47 MHz) δ (ppm)=22.82, 47.66, 55.39, 75.25, 90.07, 115.33, 115.62, 118.26, 121.15, 121.20, 121.25, 121.88, 125.49, 127.07, 127.47, 129.11, 129.19, 129.30, 129.84, 130.27, 130.70, 131.14, 132.95, 132.99, 148.05, 160.98, 162.01, 165.30.

### Example 16

### Synthesis of Iso-III

4.42 g (10 mmol, 1 equivalent) of iso-XI are dissolved in 100 mL of methanol. Then 0.88 g of (Pd/C 10%) are added. The reaction mixture is hydrogenated in 16 h using a hydrogen balloon. The catalyst is filtered of and washed three times with 25 mL of methanol. The filtrate is concentrated under reduced pressure (45° C., 10 mbar) to yield 4.38 g (98.7%) of the product iso-III as a colourless oil. The crude product is used without any purification for the next reaction step.

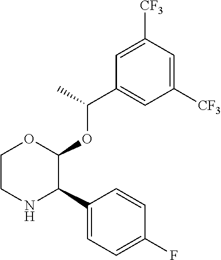

¹H-NMR (DMSO-d₆, 300 MHz) δ (ppm)=1.02 (d, CH₃, 3H, J 6.4 Hz), 2.88 (m, CH₂, 2H), 3.30 (dd, CH₂, 1H, J 10.6 Hz, J 1.9 Hz), 3.63 (dt, CH₂, 1H, J 11.0 Hz, J 3.6 Hz), 4.00 (d, CH, 1H, J 2.3 Hz), 4.78 (q, CH, 1H, J 6.4 Hz), 4.91 (d, CH, 1H, J 2.6 Hz), 7.12 (t, CH, 2H, J 8.9 Hz), 7.49 (m, CH, 2H), 7.92 (s, CH, 1H), 7.95 (s, CH, 2H).

¹³C-NMR (DMSO-d₆, 300 MHz) δ (ppm)=21.97, 45.73, 59.39, 61.41, 73.22, 96.08, 114.42, 114.70, 118.28, 120.90, 120.95, 121.00, 121.89, 125.51, 126.88, 126.91, 129.12, 129.49, 129.60, 129.77, 130.20, 130.63, 131.07, 136.89, 136.93, 148.24, 159.90, 163.11.

### Example 17

### Synthesis of Iso-I

N-[1-Amino-2-chloro-eth-(Z)-ylidene]-hydrazinecarboxylic acid methyl ester-variant:

3.80 g (8.71 mmol, 1 equivalent) iso-III are dissolved in 32 mL acetonitrile, before 4.29 mL (25.07 mmol, 2.9 equivalents) N,N-Diisopropylethylamine and 1.86 g (11.23 mmol, 1.29 equivalents) N-[1-Amino-2-chloro-eth-(Z)-ylidene]-hydrazinecarboxylic acid methyl ester are added. The suspension is stirred for 3 h, while a clear solution is formed. The reaction mixture was concentrated under reduced pressure (45° C., 100 mbar) and the residue was dissolved in 100 mL dichloromethane and washed with 100 mL of a 26.5% aqueous sodium chloride solution. The organic layer is concentrated under reduced pressure. Then 34 mL acetonitrile are added. The mixture is transferred to a reactor where it is stirred for 24 h at 95° C. and 1.5 bar. The reaction mixture is concentrated under reduced pressure (45° C., 10 mbar) and the residue is dissolved in 42 mL methanol. The reaction mixture is brought to reflux and a spoonful of charcoal is added. The reaction mixture is stirred at this temperature for 30 min. The product is filtered and the filter is washed with methanol. The product is concentrated under reduced pressure. The reaction yields 4.94 g (100%) of a slightly yellow crystalline product iso-I.

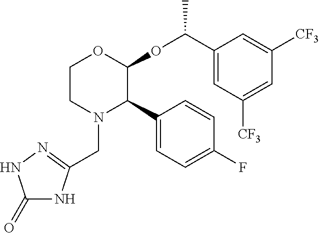

NMR: ¹H-NMR (DMSO-d₆, 300 MHz) δ (ppm)=0.98 (d, CH₃, 3H, J 6.3 Hz), 2.33 (dt, CH₂, 1H, J 11.4 Hz, J 2.7 Hz), 2.73 (d, CH₂, 1H, J 11.6 Hz), 2.84 (d, CH₂, 1H, J 13.9 Hz), 3.40 (m, CH₂, 3Hz), 3.85 (t, CH₂, 1H, J 6.9 Hz), 4.65 (q, CH, 1H, J 6.1 Hz), 4.85 (d, CH, 1H, J 2.6 Hz), 7.14 (t, CH, 2H, J 8.8 Hz), 7.60 (dt, CH, 2H, J 6.0 Hz, J 2.1 Hz), 7.91 (s, CH, 2H), 7.95 (s, CH, 1H), 11.29 (s, NH, 1H), 11.41(bs, NH, 1H).

¹³C-NMR (DMSO-d₆, 75.47 MHz) δ (ppm)=22.28, 51.17, 51.57, 60.12, 68.32, 74.25, 97.33, 115.12, 115.40, 121.67, 122.39, 126.00, 127.49, 129.61, 130.27, 130.71, 131.14, 131.57, 131.87, 132.27, 132.38, 134.34, 144.63, 148.51, 157.12, 160.79, 164.01.

1. A morpholine derivative of formula VII or formula iso-VII as an addition salt with a chiral acid wherein R₁ is benzyl, substituted benzyl or another nitrogen protecting group.

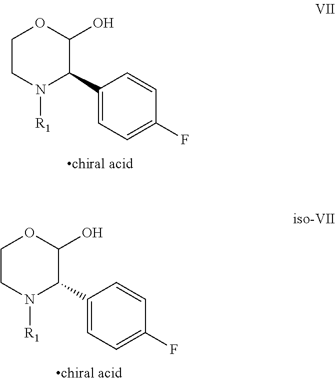

2. The compounds of claim 1, wherein the chiral acid is selected from the group consisting of tartaric acid or tartaric acid derivatives, camphersulfonic acid derivatives such as 3-bromocamphor-10-sulfonic acid, camphanic acid, 10-camphorsulfonic, or camphoric acid, amino acids such as glutamic acid, valine, or aspartic acid, mandelic acid or mandelic acid derivatives such as α-methoxy-α-trifluoromethylphenylacetic or α-methoxyphenylacetic acid, acetoxy-5-etienic acid, malic acid, menthyloxyacetic acid, N-(α-methylbenzyl)succinamidic acid, N-[1-(1-naphthyl)ethyl]succinamic acid, N-(1-phenylethyl)succinamic acid, 1-mono-menthyl phthalate, N,N-Bis[1-phenylethyl]phthalamic acid, N-(1-phenylethyl)phthalamic acid, 2-phenylpropionic acid, phenylcarbamoyloxypropionic acid, pyroglutamic acid, quinic acid, 1,4-benzodioxane-2-carboxylic acid, 1,1′-binaphthalene-2,2′-diyl hydrogen phosphate, or 5-oxo-2-tetrahydrofurancarboxylic acid in either enantiomeric or diastereomeric form.

3. The compounds of claims 1-2, wherein the chiral acid is tartaric acid or a tartaric acid derivative such as di-O,O′-toluoyl tartaric acid, di-O,O′-benzoyl tartaric acid, di-O,O′-anisoyl tartaric acid, or O,O′-dibenzoyl tartaric acid mono(dimethylamide).

4. The compounds of claims 1-3, wherein the tartaric acid derivative is di-O,O′-toluoyl tartaric acid.

5. The compound of formula VII according to claim 1, wherein the chiral acid is L-di-O,O′-toluoyl tartaric acid and R₁ is benzyl.

6. The compound of formula iso-VII according to claim 1, wherein the chiral acid is D-di-O,O′-toluoyl tartaric acid and R₁ is benzyl.

7. A process for the preparation of morpholine derivatives according to claim 1 comprising the steps of
coupling an amino alcohol of formula VI, 4-fluorophenylboronic acid or a C₁₋₆ alkyl or cyclic ester thereof, and glyoxal
crystallization of the obtained morpholine derivative acid as an addition salt with a chiral acid
isolation of a product of formula VII.chiral acid or of formula iso-VII.chiral acid
optionally razemizing the undesired isomer and resubjecting the razemized morpholine derivative to a crystallization with a chiral acid.

8. The process of claim 7, wherein the chiral acid is selected from the group consisting of tartaric acid or tartaric acid derivatives, camphorsulfonic acid derivatives such as 3-bromocamphor-10-sulfonic acid, camphanic acid, 10-camphorsulfonic, or camphoric acid, amino acids such as glutamic acid, valine, or aspartic acid, mandelic acid or mandelic acid derivatives such as α-methoxy-α-trifluoromethylphenylacetic or α-methoxyphenylacetic acid, acetoxy-5-etienic acid, malic acid, menthyloxyacetic acid, N-(α-methylbenzyl)succinamidic acid, N-[1-(1-naphthyl)pethyl]succinamic acid, N-(1-phenylethyl)succinamic acid, 1-mono-menthyl phthalate, N,N-Bis[1-phenylethyl]phthalamic acid, N-(1-phenylethyl)phthalamic acid, 2-phenylpropionic acid, phenylcarbamoyloxypropionic acid, pyroglutamic acid, quinic acid, 1,4-benzodioxane-2-carboxylic acid, 1,1′-binaphthalene-2,2′-diyl hydrogen phosphate, or 5-oxo-2-tetrahydrofurancarboxylic acid in either enantiomeric or diastereomeric form.

9. The process of claims 7-8, wherein the chiral acid tartaric acid or a tartaric acid derivative such as di-O,O′-toluoyl tartaric acid, di-O,O′-benzoyl tartaric acid, di-O,O′-anisoyl tartaric acid, or O,O′-dibenzoyl tartaric acid mono(dimethylamide).

10. The process of claims 7-9, wherein the tartaric acid derivative is di-O,O′-toluoyl tartaric acid.

11. The process of claim 7, wherein the isolated product is of formula VII.L-di-O,O′-toluoyl tartaric acid or formula iso-VII.D-di-O,O′-toluoyl tartaric acid.

12. The process of claim 11, wherein R₁ is benzyl or substituted benzyl.

13. Use of the compound of formula VILL-di-O,O′-toluoyl tartaric acid in the synthesis of aprepitant or fosaprepitant.

14. Use of the compound of formula iso-VII.D-di-O,O′-toluoyl tartaric acid in the synthesis of the compounds of formula iso-I or iso-II.

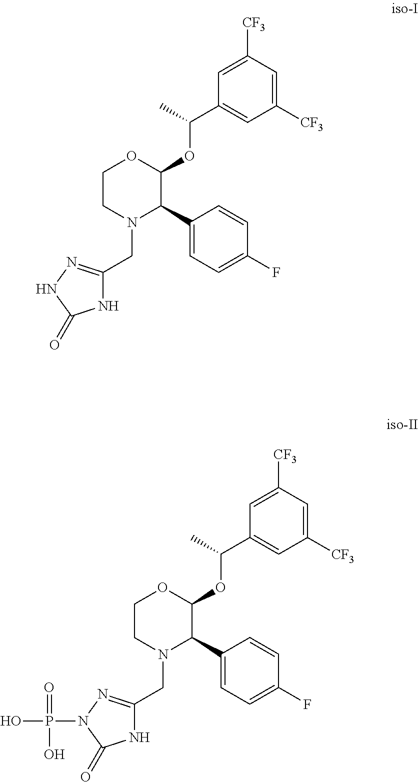

15. A process for the preparation of aprepitant (I) or fosaprepitant (II), comprising the steps of
a) a three component coupling of an amino alcohol of formula VI, of 4-fluorophenylboronic acid or a C₁₋₆ alkyl or cyclic ester thereof (formula V), and of glyoxal (IV) followed by crystallization of the obtained morpholine derivative as addition salt with a chiral acid and isolation of a product of formula VII.chiral acid;
b) generation of hemi-acetal VII free base by portioning compound of formula VII.chiral acid between an alkaline aqueous layer and a water-immiscible organic phase; activation of the hemi-acetal functionality by transforming the OH-group into an activated derivative; reaction of the resulting activated acetal with alcohol of formula VIII. Removal of impurities by extraction to get a solution of compound IX;
c) removal of the N-protecting group to give the amine of formula X;
d) oxidation of the amine of formula X to the corresponding cyclic imine of formula X and isolation of the cyclic imine of formula XI;
e) reduction of imine XI with a catalyst and H₂ or an H₂ equivalent; removal of the catalyst by filtration to get the key intermediate of formula III;
f) alkylation of compound of formula III to give aprepitant or fosaprepitant directly or via e.g. protected intermediates;
g) optionally, conversion of aprepitant to fosaprepitant by phosphorylation or a phosphorylation-deprotection sequence.

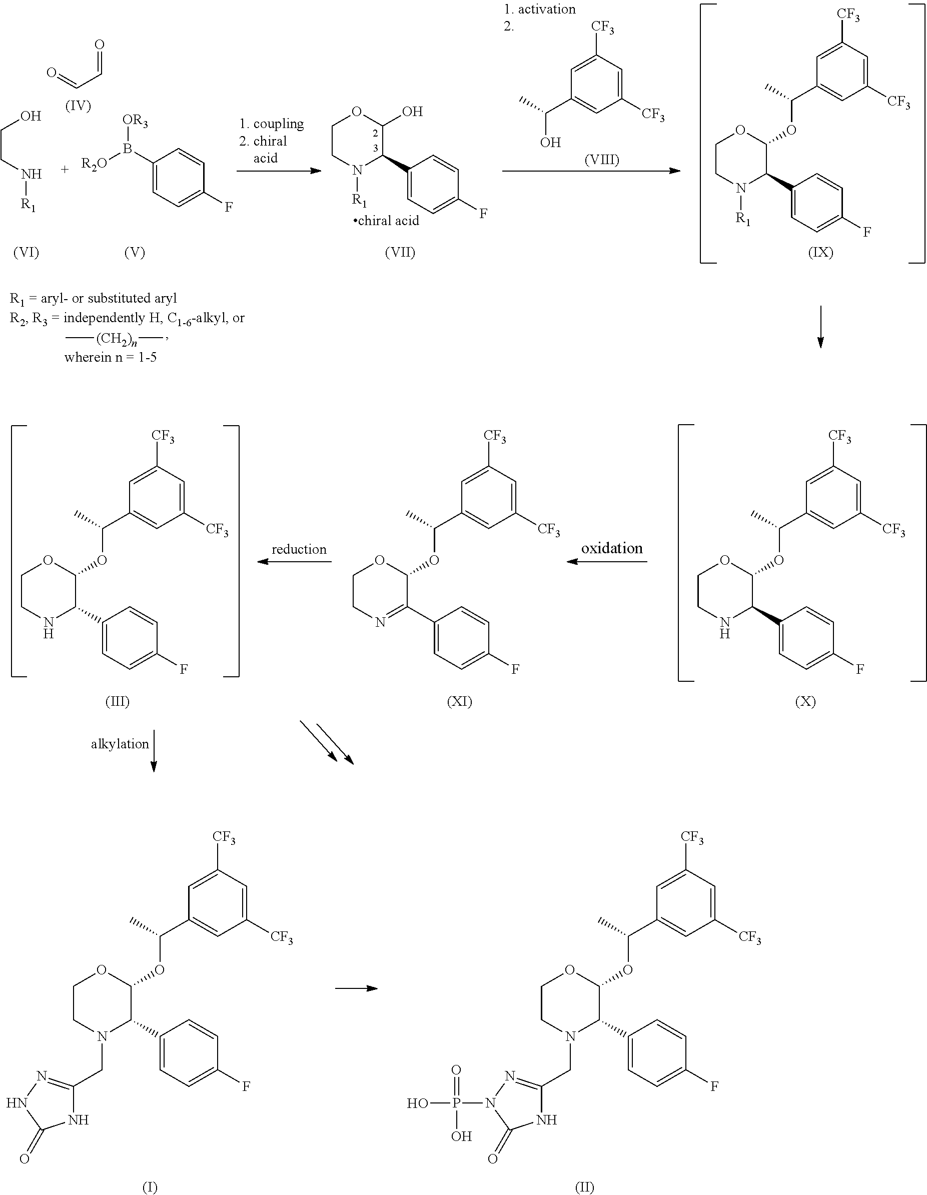

16. The process according to claim 15, characterized in that the isolated intermediates are the compounds of formula VII.chiral acid and of formula XI.

17. A process for the preparation of a compound of formula iso-I or iso-II, comprising the steps of
a) a three component coupling of an amino alcohol of formula VI, of 4-fluorophenylboronic acid or a C₁₋₆ alkyl or cyclic ester thereof (formula V), and of glyoxal (IV) followed by crystallization of the obtained morpholine derivative as addition salt with a chiral acid and isolation of a product of formula iso-VII.chiral acid;
b) generation of hemi-acetal iso-VII free base by portioning compound of formula iso-VII.chiral acid between an alkaline aqueous layer and a water-immiscible organic phase; activation of the hemi-acetal functionality by transforming the OH-group into an activated derivative; reaction of the resulting activated acetal with alcohol of formula VIII. Removal of impurities by extraction to get a solution of compound iso-IX;
c) removal of the N-protecting group to give the amine of formula iso-X;
d) oxidation of the amine of formula iso-X to the corresponding cyclic imine of formula iso-X and isolation of the cyclic imine of formula iso-XI;
e) reduction of imine of formula iso-XI with a catalyst and H₂ or an H₂ equivalent; removal of the catalyst by filtration to get the key intermediate of formula iso-III;
f) alkylation of compound of formula iso-Ill to give a compound of formula iso-I or of formula iso-II directly or via e.g. protected intermediates;
g) optionally, conversion of a compound of formula iso-I to a compound of formula iso-II by phosphorylation or a phosphorylation-deprotection sequence.

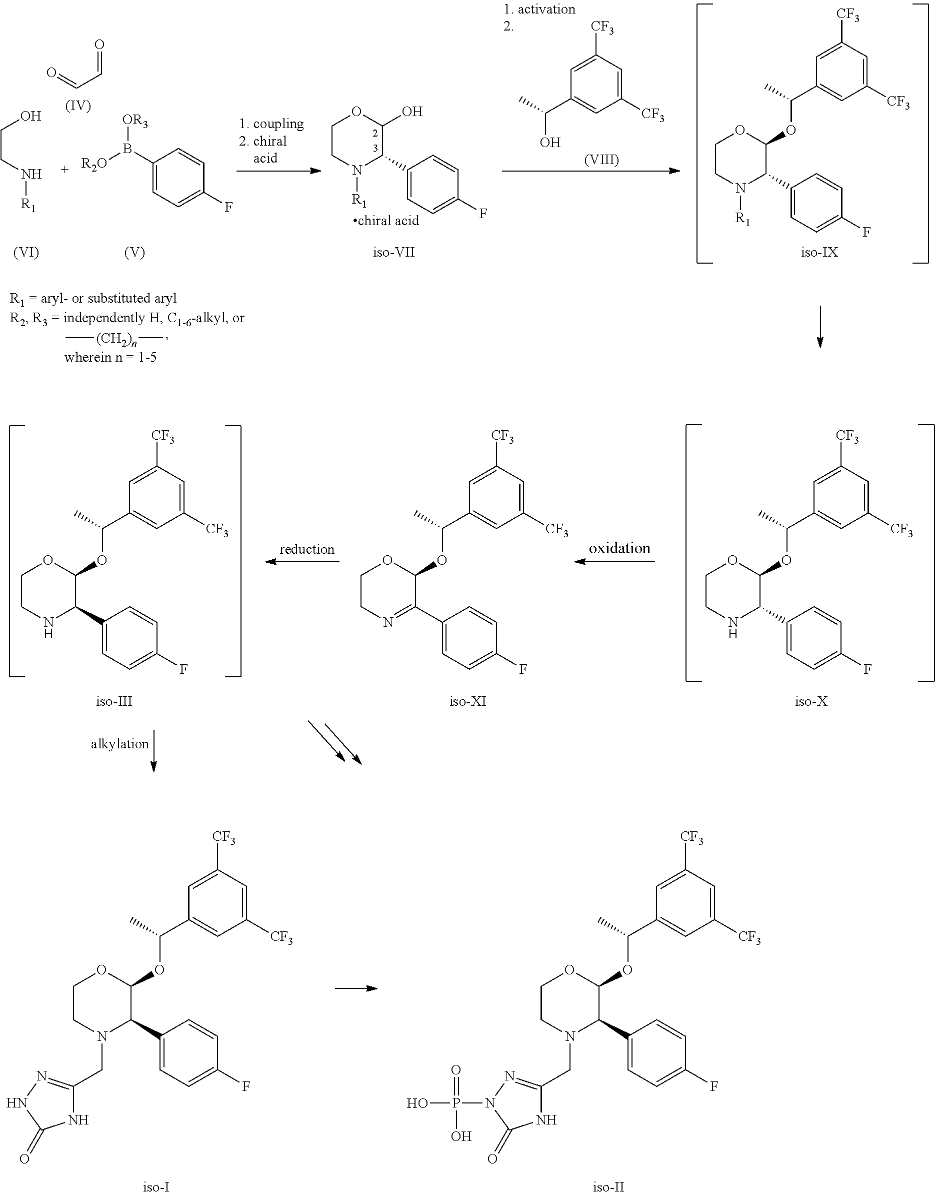

18. A compound of formula iso-XI.

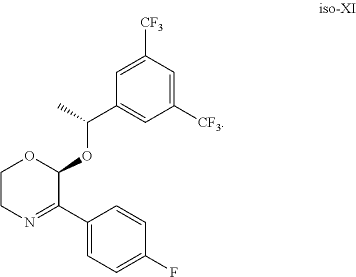

19. A compound of formula iso-I.

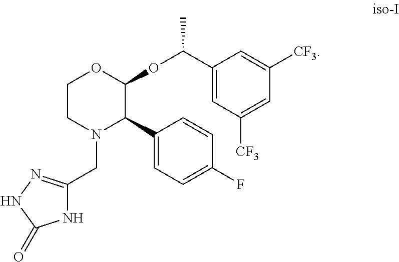

20. A compound of formula iso-II. [chemical structure]
